(19) United States
(12) Patent Application Publication
(10) Pub. No.: US 2025/0163403 A1
(43) Pub. Date: May 22, 2025

(54) METHOD OF INTRODUCING A PLURALITY OF GENE DRIVES INTO A POPULATION

(71) Applicant: THE PIRBRIGHT INSTITUTE, Woking (GB)

(72) Inventors: Luke Alphey, Woking Surey (GB); Tim Harvey-samuel, Woking Surey (GB); Matt Edgington, Woking Surey (GB)

(21) Appl. No.: 17/607,138

(22) PCT Filed: May 1, 2020
(86) PCT No.: PCT/GB2020/051069

§ 371 Date: Oct. 28, 2021

(30) Foreign Application Priority Data
May 2, 2019 (GB) 1906203.3

(51) Int. Cl. C12N 15/10; C12N 9/22; C12N 15/11; C12N 15/90

(52) U.S. Cl. CPC C12N 15/102; C12N 9/22; C12N 15/111; C12N 15/902; C12N 2310/20

(57) ABSTRACT

The present invention relates to a method of generating a plurality of engineered subpopulations each comprising a gene drive.

### FIELD OF THE INVENTION

The present invention relates to a method of introducing a plurality of gene drives into a population. In particular, the present invention relates to methods to manage resistance to a gene drive.

### BACKGROUND TO THE INVENTION

The modification of wild populations using gene drive systems has been proposed as a method for managing global issues such as agricultural pests, invasive species and the spread of disease from mosquito-borne pathogens.

Multiple types of gene drive systems have been proposed, all of which are intended to spread modified genomic sequences to a high frequency in a population beyond what standard Mendelian inheritance would normally allow. Importantly, gene drives can spread within a population, even if they confer a fitness cost on individuals carrying them.

The most well-known type of gene drive is the ‘homing drive’, which introduces double-stranded breaks (DSBs) at a specific sequence within a wild-type locus homologous to the drive transgene. Examples of homing drives include the CRISPR-Cas9 system, whereby the Cas9 endonuclease is directed to a target site by a complementary guide RNA (gRNA) and can introduce a DSB. The gene drive construct itself is then ideally used to repair the DSB, in a process known as homology-directed repair (HDR), whereby the gene drive construct is copied onto the homologous chromosome. This process allows a gene drive to spread rapidly within a population.

However, DSBs can also be repaired by other mechanisms, such as non-homologous end-joining (NHEJ), whereby broken ends of DNA are ligated back together. Issues arise with this repair mechanism when insertion or deletion mutations are introduced which alter DNA sequences at target sites, thereby making these sites resistant to recognition by further homing drives. So called cut-resistant alleles significantly impair the spread of homing drives in a population, as resistance alleles that confer a fitness advantage relative to the gene drive transgene are likely to spread, eventually leading to loss of the gene drive system.

To overcome the issue of resistance, one proposed strategy is to engineer ‘multiplexing’ gene drives that target multiple linked sites of a single locus through the simultaneous expression of several different gRNAs. Thus, resistance could only then occur when cut-resistance alleles develop at all of the gRNA target sites, preventing the drive from targeting the homologous chromosome further.

However, a problem that arises from this ‘multiplexing’ strategy is that these simultaneous DSBs at multiple sites on the same chromosome could be repaired by mechanisms other than HDR, such as NHEJ or micro-homology-dependent end-joining, which could result in the deletion of the entire intervening sequence, effectively creating resistance at all target sites between those DSBs.

Issues of resistance are particularly important for gene drives targeting a neutral locus (i.e. a locus that doesn't have substantial fitness cost such as an intergenic or non-coding locus) because drive-resistant deletion mutants will likely retain full fitness.

Since the power of gene drive systems is that they spread ‘cargo genes’ within a population, such as malaria-refractory genes in mosquitoes and suppressive sex-ratio biases in invasive rodents, it is imperative that the spread of gene drive systems are not impaired by drive-resistance mutations.

Therefore a need exists to develop alternative resistance management strategies to improve on existing gene drive systems.

### SUMMARY OF THE INVENTION

The present invention is based on the provision of at least two gene drives which multiplex at the population level. In particular, the present invention is based on providing individual nuclease targeting entities directed against different target sites in a target locus such that separate gene drive constructs are capable of segregating independently throughout a population.

Importantly, the separate gene drive constructs, and in particular the nuclease targeting entities, have a functional interaction. As such, in the event that two of the present gene drive constructs directed against a particular target locus are inherited by an individual within the population, each of the individual gene drive constructs is capable of influencing the ability of the other gene drive to copy and thus an individual has a reduced likelihood of retaining more than one gene drive construct per haploid cell (e.g. gamete).

For example, in one embodiment, when an individual from a first engineered subpopulation comprising a first gene drive mates with an individual from a second engineered subpopulation comprising a second gene drive to produce a germline cell comprising both the first gene drive and the second gene drive; each of the first gene drive and the second gene construct may be capable of replacing the other gene drive construct on the sister chromosome.

In another embodiment, when an individual from a first engineered subpopulation comprising a first gene drive mates with an individual from a second engineered subpopulation comprising a second gene drive to produce a germline cell comprising both the first gene drive and the second gene drive; each of the first gene drive and the second gene constructs are incapable of cleaving the other gene drive construct on the sister chromosome. As such, multiple double-strand breaks are avoided.

The same principles are maintained if more than two (for example 3, 4, 5 or more) gene drive constructs are introduced into separate subpopulations. For example, (i) either each gene drive construct is associated with a nuclease targeting entity which is capable of directing a homing nuclease to cut the exogenous nucleotide sequence in each of the other gene drive constructs (such that each gene drive construct is capable of cutting and copying to replace any of the other gene drive constructs in a sister chromosome); or (ii) none of the gene drive constructs is associated with a nuclease targeting entity which is capable of directing a homing nuclease to cut the exogenous nucleotide sequence in any of the other gene drive constructs (such that each gene drive construct is incapable of cutting and copying to replace any of the other gene drive constructs in a sister chromosome).

Because each gene drive construct is capable of influencing the activity of the other, the genome of an individual within a population is less likely to be subject to deletion of the entire intervening sequence and effective resistance at all target sites between DSBs.

However, because there are at least two gene drive constructs present within the population, the gene drives will still be spread at the population level. In addition, the generation of a cut-resistant allele will require a separate insertion or deletion mutation for each individual gene drive target site in the target locus. Thus the probability of generating sub-populations that are resistant to each of the gene drive constructs present in the population is greatly reduced compared to a classical multiplexing strategy.

Accordingly, in the present invention the creation of multiple simultaneous DSBs on the same chromosome is essentially eliminated as each construct only carries a single nuclease targeting entity and the constructs have a reduced likelihood to become linked.

Thus, in a first aspect the present invention provides a method of generating a plurality of engineered subpopulations each comprising a gene drive, said method comprising:
introducing a first gene drive construct into the germline genomic DNA of a first subpopulation to produce a first engineered subpopulation, which first gene drive construct comprises a first exogenous nucleotide sequence comprising a first entity which is a component of a homing nuclease; which homing nuclease is capable of a targeting a first target sequence via a first targeting entity, and a first flanking sequence and a second flanking sequence, wherein at least the first exogenous nucleotide sequence is located between the first flanking sequence and the second flanking sequence;
introducing a second gene drive construct into the germline genomic DNA of a second subpopulation to produce a second engineered subpopulation, which second gene drive construct comprises a second exogenous nucleotide sequence comprising a second entity which is a component of a homing nuclease; which homing nuclease is capable of a targeting a second target sequence via a second targeting entity, and a third flanking sequence and a fourth flanking sequence, wherein at least the second exogenous nucleotide sequence is located between the third flanking sequence and the fourth flanking sequence;
wherein the first and second flanking sequences and the third and fourth flanking sequences are substantially identical to target regions located in the same target locus in the genomic DNA;
and wherein each of the first target sequence and the second target sequence in the genomic DNA is located between the target regions which are substantially identical to the first and second flanking sequences and the third and fourth flanking sequences or within a target region which is substantially identical to one of the first, second, third or fourth flanking sequence;
wherein
   (i)
(a) the first exogenous nucleotide sequence between the first flanking sequence and second flanking sequence of the first gene drive comprises a target sequence for the second nuclease targeting entity; and
(b) the second exogenous nucleotide sequence between the third flanking sequence and fourth flanking sequence of the second gene drive comprises a target sequence for the first nuclease targeting entity; or
   (ii)
(a) the first exogenous nucleotide sequence between the first flanking sequence and second flanking sequence of the first gene drive does not comprise a target sequence for the second targeting entity; and
(b) the second exogenous nucleotide sequence between the third flanking sequence and fourth flanking sequence of the second gene drive does not comprise a target sequence for the first nuclease targeting entity.

In the embodiment described for part (i) of the present method: when an individual from the first engineered subpopulation mates with an individual from the second engineered subpopulation to produce a germline cell comprising the first gene drive and the second gene drive:
(A) the first nuclease targeting entity directs the homing nuclease of the first gene drive to the target sequence which is present in the exogenous nucleotide sequence of the second gene drive such that the nuclease cleaves the target sequence in a cleavage site specific manner and the first gene drive is inserted into the target location in place of the second gene drive; or
(B) the second nuclease targeting entity directs the homing nuclease of the second gene drive to the target sequence which is present in the exogenous nucleotide sequence of the first gene drive such that the nuclease cleaves the target sequence in a cleavage site specific manner and the second gene drive is inserted into the target location in place of the first gene drive.

Accordingly, in the embodiment described in part (i), each gene drive construct comprises a target sequence for each of the nuclease targeting entities of the other gene drives used in the method. As such, each gene drive construct will be targeted to the target site of its nuclease targeting entity which is present in the other gene drive construct. In addition, the exogenous nucleotide sequence of each gene drive construct comprises target sequences for the nuclease targeting entities of each of the other segregating, multiplex gene drive construct(s) introduced into the population. As such, successful homing reactions will always restore the target sites for the other segregating multiplex gene drive constructs. In other words, successful homing by any of the segregating multiplex gene drive constructs will delete any existing construct and/or resistant alleles on the homologous chromosome. This embodiment may be referred to herein as the “overwriting strategy”.

In the embodiment described for part (ii) of the present method: when an individual from the first engineered subpopulation mates with an individual from the second engineered subpopulation to produce a germline cell comprising the first gene drive and the second gene drive:
(A) the first nuclease targeting entity of the first gene drive is incapable of targeting a nuclease to the second gene drive because the second gene drive does not comprise a target sequence for the nuclease targeting entity of the first gene drive; and
(B) the second nuclease targeting entity of the second gene drive is incapable of targeting a nuclease to the first gene drive because the first gene drive does not comprise a target sequence for the nuclease targeting entity of the second gene drive.

As such, none of the exogenous nucleotide sequences in the gene drive constructs comprises a target site for any of the nuclease targeting entities of the other gene drive constructs used in the method. For example, the first gene drive is incapable of targeting a nuclease to cleave the exogenous nucleotide sequence of the second gene drive; and the nuclease targeting entity of the second gene drive is incapable of targeting a nuclease to cleave the exogenous nucleotide sequence of the first gene drive. In other words, a successful homing event by one construct removes the target sites of the other segregating gene drive constructs from the genome of an individual. This embodiment may be referred to herein as the “blocking strategy”.

Illustrative applications for the methods and constructs of the present invention include, for example, as elements in daisy chain drives (Noble, C., et al. (2019), PNAS, 16 (17): 8275-8282).

In particular, the methods and constructs of the present invention may be used as elements of daisy chains where the target locus is a neutral locus or a locus which is not fertility- or viability-essential.

### DESCRIPTION OF FIGURES

FIG. 1—A schematic of the possible repair mechanisms (HDR and NHEJ) when two DSBs are induced simultaneously by a multiplexed gene drive system. For HDR, the whole multiplexed construct homes into the homologous chromosome and removes target sites due to the location of homology arms. In the case of NHEJ the intervening sequence between the DSBs is deleted and the two ends ligated together resulting in resistance at these target sites (and all in between).

FIG. 2—Schematic of embodiments of the invention. In the overwriting strategy (a), constructs are designed such that successful homing of a single construct results in restoration of the other target sites (i.e. any constructs or resistant alleles on the homologous chromosome are replaced by the homed construct and a set of intact target sites for the other constructs). The blocking strategy (b) functions similarly but instead of restoring missing target sites this strategy deletes all other target sites on the homologous chromosome.

FIG. 3—Representative numerical simulations of a CRISPR-based gene drive targeting n-sites at a neutral locus through a classical multiplexing strategy. Each line represents a single simulation with colours denoting the number of sites targeted. For comparison, black lines represent a single target site CRISPR drive. In each case a release ratio of 0.05 is considered.

FIG. 4—Representative numerical simulations of a CRISPR gene drive targeting n-sites at a neutral locus through an overwriting strategy. Each line represents a single simulation with colours denoting the number of sites targeted. For comparison, black lines represent a single target site CRISPR drive. In each case a release ratio of 0.05 is considered.

FIG. 5—Representative numerical simulations of a CRISPR gene drive targeting n-sites at a neutral locus through a blocking strategy. Each line represents a single simulation with colours denoting the number of sites targeted. For comparison, black lines represent a single target site CRISPR drive. In each case a release ratio of 0.05 is considered.

FIG. 6—Illustrative embodiment of a blocking strategy in which successful homing of one gene drive results in the deletion of target sites for all of the other gene drives, illustrated here for two drive elements. (Top) Schematic diagrams of a wild-type chromosome and example structures for two blocking gene drive constructs. (Middle) An example of the desired mechanism of action when a gene drive construct encounters a wild-type chromosome. In the first instance Guide RNA #1 directs the nuclease to cut the wild-type chromosome at Target Site #1. The sequence between recognised regions of homology is then resected, thus deleting other target sites and allowing the gene drive to be used as a repair template resulting in an individual homozygous for the gene drive. (Bottom) In the case where two gene drives encounter one another there is no effect since none of the guide RNAs is able to find its associated target site.

FIG. 7—Illustrative embodiment of a blocking strategy in which successful homing of one gene drive results in the recoding of target sites for all of the other gene drives. (Top) Schematic diagrams of a wild-type chromosome and example structures for two blocking gene drive constructs. (Middle) An example of the desired mechanism of action when a gene drive construct encounters a wild-type chromosome. In the first instance Guide RNA #1 directs the nuclease to cut the wild-type chromosome at Target Site #1. The sequence between recognised regions of homology is then resected, allowing the gene drive to be used as a repair template resulting in an individual homozygous for the gene drive. The close proximity of target sites and the length of conversion tracts allows the recoded target sites to be copied over to the homologous chromosome. (Bottom) In the case where two gene drives encounter one another there is no effect since none of the guide RNAs are able to find a non-recoded version of their associated target sites.

FIG. 8—Illustrative embodiment of an overwriting strategy in which successful homing of one gene drive results in the copying of target sites for all of the other gene drives. (Top) Schematic diagrams of a wild-type chromosome and example structures for two overwriting gene drive constructs. (Middle) An example of the desired mechanism of action when a gene drive construct encounters a wild-type chromosome. In the first instance Guide RNA #1 directs the nuclease to cut the wild-type chromosome at Target Site #1. The sequence between recognised regions of homology is then resected, allowing the gene drive to be used as a repair template resulting in an individual homozygous for the gene drive (including target sites for all other gene drive constructs). Although illustrated here as complete resection of Gene Drive #1, if Gene Drive #1 and Gene Drive #2 have regions of sequence similarity, the resected region may be considerably shorter. (Bottom). An example of the effect when two gene drives encounter one another. In this example Guide RNA #2 directs the nuclease from Gene Drive #2 to cut Target Site #2 within Gene Drive #1. The sequence between recognised regions of homology is then resected, allowing Gene Drive #2 to be copied across and forming and individual homozygous for Gene Drive #2

FIG. 9—Illustrative embodiment of an overwriting strategy in which successful homing of one gene drive results in the copying of target sites for all of the other gene drives via their inclusion within the flanking sequences. (Top) Schematic diagrams of a wild-type chromosome and example structures for two overwriting gene drive constructs. (Middle) An example of the desired mechanism of action when a gene drive construct encounters a wild-type chromosome. In the first instance Guide RNA #1 directs the nuclease to cut the wild-type chromosome at Target Site #1. The sequence between recognised regions of homology is then resected, allowing the gene drive to be used as a repair template resulting in an individual homozygous for the gene drive (including target sites for all other gene drive constructs due to the length of conversion tracts). (Bottom) An example of the effect when two gene drives encounter one another. In this example Guide RNA #2 directs the nuclease from Gene Drive #2 to cut Target Site #2 within Gene Drive #1. The sequence between recognised regions of homology is then resected, allowing Gene Drive #2 to be copied across and forming and individual homozygous for Gene Drive #2.

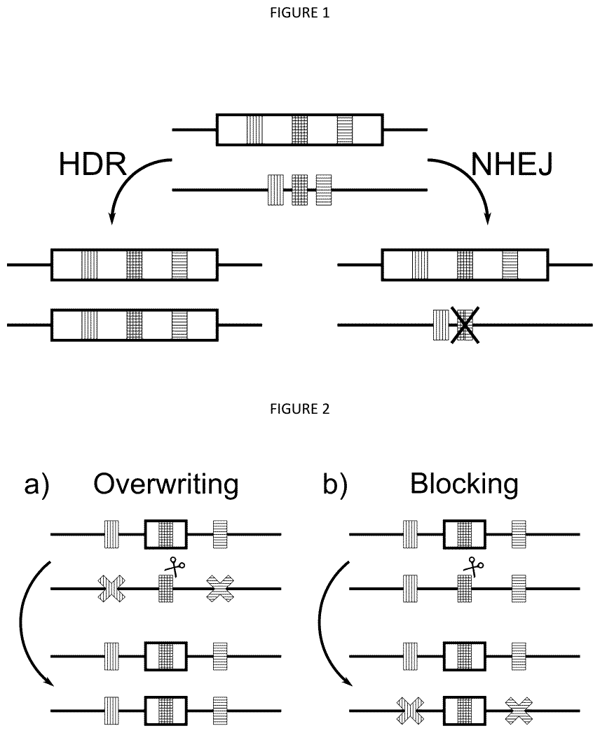

### DETAILED DESCRIPTION OF THE INVENTION

### Gene Drive

In sexually-reproducing species, most genes are present in two copies (which can be the same or different alleles), either one of which has a 50% chance of passing to a descendant. In classical Darwinian terms, for a particular allele to spread through a large population, it must increase the fitness of those organisms which carry it. However, some sequences have evolved molecular mechanisms that confer on them a greater chance of transmission. This allows them to spread through a population even if they reduce the fitness of carriers. By biasing the inheritance of particular altered genes, synthetic gene drives may spread alterations through a population.

“Gene drive” as used herein refers to a ‘homing-drive’ which is spread through a population by the action of a target-site directed nuclease and the subsequent copying of a template strand which is used to repair a double-strand break at the cutting site of the nuclease as part of a homologous recombination repair process. Importantly, the template strand is provided as an exogenous nucleotide sequence on the gene drive construct. The concept of a gene drive is known in the art—see Windbichler et al. (Nature, doi: 10.1038/nature09937; 2011) and Burt (Proc. R. Soc. Lond. B; 2003; 270; 921-928).

As such, a gene drive is capable of self-propagating through a population by a mechanism in which the target-site directed nuclease cuts a target DNA sequence at a specific site in a target locus that does not encode the drive, inducing the cell to repair the damage by copying the drive sequence onto the damaged chromosome. The cell then has two copies of the drive sequence. As such a gene drive—and in particular an exogenous nucleotide sequence—is stably introduced in to the germline cell of a desired organism.

A resulting transgenic organism may be developed from the germline cell. The resulting transgenic organism can then be introduced into a wild type population, and through mating by the transgenic organism with a wild type organism, the exogenous nucleic acid sequence is transferred to resulting offspring or progeny. The methods of the present invention are thus directed to the production of a population of transgenic organisms having desired traits from one of a plurality of initial transgenic organisms and a wild type organism.

The gene drive of the present invention may comprise an exogenous nucleotide sequence located between two flanking sequences.

The exogenous nucleotide sequence comprises, or encodes, a first entity which is a component of a homing nuclease.

Suitably, the component of the homing nuclease may be a targeting entity which is capable of targeting a nuclease to a target sequence and/or a nuclease.

The entity which is capable of a targeting a nuclease to a target sequence may be a polynucleotide which is capable of directing a nuclease to a target site, a polynucleotide encoding a polypeptide which is capable of directing a nuclease to a target site or a polynucleotide which encodes a target-specific nuclease.

The exogenous nucleotide sequence may comprise a nucleic acid sequence which is, or encodes, a nuclease targeting entity. The exogenous nucleotide sequence may comprise a nucleic acid sequence which encodes a nuclease.

The exogenous nucleotide sequence may comprise a nucleic acid sequence which is, or encodes, a nuclease targeting entity and a nucleic acid sequence which encodes a nuclease.

The exogenous nucleotide sequence also may comprise at least one promoter such that the component of the homing nuclease can be expressed. The particular promoter will depend on the target organism. The promoter may be a constitutive promoter or an inducible promoter. The choice of a suitable promoter is routine in the art. The exogenous nucleotide sequence may further comprise any other nucleic acid sequences required for expression of the nucleic acid sequences provided by the exogenous nucleotide sequence, in particular in a germline cell.

The exogenous nucleotide sequence may also comprise any further nucleic acid sequences which it is desired to be expressed in the cell, in particular in the germline cell. Such further nucleic acid sequences may be referred to as “cargo nucleic acid”. It is within the ambit of the skilled person to provide suitable cargo nucleic acid to achieve the desired trait. For example, the cargo nucleic acid may comprise a polypeptide-encoding sequence which is to be introduced into a population. Alternatively, the cargo nucleic acid may comprise a mutant copy of the corresponding wild type sequence present in the genome of the target population. The mutant may, for example, comprise a deletion, addition or substitution compared to the corresponding wild type sequence.

The exogenous nucleotide sequence may be inserted into a gene in the target genome, such that the function or activity of that gene is reduced, disrupted or deleted.

As described herein, in the “overwriting” embodiment the exogenous nucleotide sequence may also comprise a target site for each of the other gene drives of the multiplex gene drive system. For example, if the method comprises introducing a first gene drive into the germline genomic DNA of a first subpopulation and introducing a second gene drive into the germline genomic DNA of a second subpopulation; the exogenous nucleotide sequence of the first gene drive may comprise a target site for the nuclease targeting entity of the second gene drive and the exogenous nucleotide sequence of the second gene drive may comprise a target site for the nuclease targeting entity of the first gene drive.

If the method comprises introducing a first gene drive into the germline genomic DNA of a first subpopulation; a second gene drive into the germline genomic DNA of a second subpopulation, and a third gene drive into the germline genomic DNA of a third subpopulation; the exogenous nucleotide sequence of the first gene drive may comprise a target site for the nuclease targeting entity of the second gene drive and the nuclease targeting entity of the third gene drive; the exogenous nucleotide sequence of the second gene drive may comprise a target site for the nuclease targeting entity of the first gene drive and the nuclease targeting entity of the third gene drive; and the exogenous nucleotide sequence of the third gene drive may comprise a target site for the nuclease targeting entity of the first gene drive and the nuclease targeting entity of the second gene drive.

Each further gene drive introduced into a further subpopulation, for example a fourth, fifth or sixth gene drive may comprise an exogenous nucleotide sequence which comprises a different nuclease targeting entity and a target site for the nuclease targeting entity of each of the other gene drive of the multiplex system.

As described herein, in the “blocking” embodiment the exogenous nucleotide sequence may not comprise a target site for one or more, in particular each, of the other gene drives of the multiplex gene drive system. For example, if the method comprises introducing a first gene drive into the germline genomic DNA of a first subpopulation and introducing a second gene drive into the germline genomic DNA of a second subpopulation; the exogenous nucleotide sequence of the first gene drive does not comprise a target site for the nuclease targeting entity of the second gene drive and the exogenous nucleotide sequence of the second gene drive does not comprise a target site for the nuclease targeting entity of the first gene drive.

If the method comprises introducing a first gene drive into the germline genomic DNA of a first subpopulation; a second gene drive into the germline genomic DNA of a second subpopulation, and a third gene drive into the germline genomic DNA of a third subpopulation; the exogenous nucleotide sequence of the first gene drive does not comprise a target site for the nuclease targeting entity of the second gene drive or the nuclease targeting entity of the third gene drive; the exogenous nucleotide sequence of the second gene drive does not comprise a target site for the nuclease targeting entity of the first gene drive or the nuclease targeting entity of the third gene drive; and the exogenous nucleotide sequence of the third gene drive does not comprise a target site for the nuclease targeting entity of the first gene drive or the nuclease targeting entity of the second gene drive.

In the “blocking” embodiment, each further gene drive introduced into a further subpopulation, for example a fourth, fifth or sixth gene drive may comprise an exogenous nucleotide sequence which comprises a different nuclease targeting entity; but the exogenous nucleotide sequence does not comprise a target site for the nuclease targeting entity of each of the other gene drive of the multiplex system.

As used herein, an exogenous nucleotide sequence means a sequence which differs from the corresponding sequence of the wild-type genome. The exogenous sequence may differ from the wild-type sequence by at least 1, at least 5, at least 10, at least 20, at least 50, at least 100, at least 500, at least 1000, at least 2000 or at least 10000 nucleotides.

As described herein, the exogenous nucleotide sequence may include an entity which is capable of targeting a nuclease to a target site.

Exogenous polynucleotides, such as the gene drive constructs of the present invention, can be introduced into a cell using any method in the art which is suitable for such introduction. Suitable methods include, for example, transfection, transduction, transformation, viral transduction, microinjection, lipofection, nucleofection, nanoparticle bombardment and conjugation.

### Engineered Subpopulation

The present invention relates to generating a plurality of engineered subpopulations, wherein each engineered subpopulation comprises a separate gene drive of the present invention.

Suitably, a plurality may be 2 or more, 3 or more, 4 or more, 5 or more, or 6 or more.

The invention may comprise preparing a plurality of engineered subpopulations as described herein and introducing them into a general population (e.g. a wild type population).

The engineered subpopulation, and general population to be targeted includes, but is not limited to, insects, mammals, plants, and fungi.

Suitably, the term mammal is used to refer to a non-human mammal.

### Germline Genomic DNA

Germline cells of the present disclosure include any germline cell into which a gene drive is to be introduced and expressed as described herein. Suitable germline cells include, but are not limited to, eukaryotic germline cells, prokaryotic germline cells, insect germline cells, mammalian germline cells, plant germline cells, fungal germline cells.

The germline cell may be homozygous or heterozygous for the gene drive construct comprising the exogenous nucleotide sequence. Suitably, the germline cell may be homozygous for the gene drive construct, for example when the target locus is a non-coding DNA sequence or a putatively non-functional locus; is not a fertility- or viability-essential locus; and/or is a fitness-neutral locus.

Suitably, the gene drive construct may be introduced into a zygote, a gamete or a cell that can give rise to a gamete.

### Nuclease Targeting Entity/Nuclease

The nuclease used herein may be any suitable homing nuclease which requires a long recognition sequence for productive cleavage. In other words the nuclease preferably only cleaves at one site in the genome of the target population.

The nuclease targeting entity targets the nuclease to a single target site in the target locus. Suitably, the nuclease targeting entity does not target the nuclease to more than one target site in the target locus.

Suitable nucleases (or nuclease systems) include, but are not limited to, RNA-guided nucleases (e.g. CRISPR-nuclease), homing endonucleases, TALENs and zinc-finger nucleases.

The nuclease may induce double-strand DNA breaks or single-strand DNA breaks (also referred to as a nickase).

The nuclease activity (e.g. the nuclease enzyme) or the nuclease targeting entity may not be encoded by the gene drive construct and may be encoded by a separate polynucleotide.

Suitably, the exogenous sequence of the gene drive construct may comprise a polynucleotide sequence which is, or encodes, a nuclease targeting entity.

Suitably, the exogenous sequence of the gene drive construct may comprise a polynucleotide sequence which encodes the nuclease.

Preferably the exogenous sequence of the gene drive construct comprises a polynucleotide sequence which is, or encodes, a nuclease targeting entity and a polynucleotide sequence which encodes the nuclease.

The nuclease targeting entity may be any entity which confers target specificity on the nuclease. The nuclease targeting entity may be a polynucleotide which is capable of directing a nuclease to a target site, a polynucleotide encoding a polypeptide which is capable of directing a nuclease to a target site or a polynucleotide which encodes a target-specific nuclease.

In embodiments in which the nuclease is a RNA-guided nuclease (e.g. CRISPR-nuclease), the nuclease targeting entity may be any RNA which confers target specificity on the nuclease. For example, the nuclease targeting entity may be a CRISPR-RNA (crRNA) or a single-guide RNA (sgRNA). The RNA may be between about 100 to about 500 nucleotides. The RNA may be between about 20 to about 100, or about 20 to about 50 nucleotides.

Suitable RNA-guided nucleases are known in the art and include Type II CRISPR nucleases.

Suitable RNA-guided nucleases include, for example, Cas9, Cpf1 or Csm1 nucleases.

Suitably, the exogenous nucleotide sequence may comprise nucleic acid sequences which between them encode all the elements necessary to provide a functional RNA-guided nuclease which cleaves at the target site of the nuclease targeting entity (i.e. the crRNA or sgRNA). For example, the exogenous nucleotide sequence may comprise a nucleic acid sequence encoding a gRNA or a crRNA and a nucleic acid sequence encoding an RNA-guided nuclease. The exogenous nucleotide sequence may comprise a nucleic acid sequence encoding a gRNA and a nucleic acid sequence encoding an RNA-guided nuclease.

Suitably, the exogenous nucleotide sequence may comprise a nucleic acid sequence encoding a homing endonuclease, TALEN or zinc-finger nuclease.

### Target Sequence

“Target sequence”, as used herein, may be synonymous with the term “target site”.

The target sequence/target site may be any nucleic acid sequence to which a nuclease targeting entity as described herein directs a nuclease to either cut or nick the genomic DNA. In the context of the present disclosure, each target site is located within the target locus of the genomic DNA of the target population.

The target site may be located within the target locus of the wild-type genomic DNA of the target population. The target site may be located with a target locus associated with a resistance allele; for example an insecticide-resistance allele or within the exogenous nucleotide sequence of another gene drive construct.

The target site may be present within a gene drive construct as described herein.

The target site may refer to the nucleic acid sequence to which the nuclease targeting entity and nuclease bind or the nucleic acid sequence at which the nuclease cleaves or nicks following targeting by the nuclease targeting entity.

By way of example, the target site of a CRISPR-nuclease is typically about 20 nucleotides in length and is selected based on at least the following criteria: (i) the sequence is unique compared to the rest of the genome; and (ii) the target site is adjacent to a Protospacer Adjacent Motif (PAM) sequence.

Approaches and techniques for selecting appropriate target sites are known in the art. Suitably PAM sequences for different CRISPR-nucleases are described in Karvelis et al. (Methods. 2017 May 15; 121-122:3-8) (hereby incorporated by reference).

Suitably, the target site of each nuclease targeting entity used in the present method should be unique and non-overlapping. In other words, the nuclease targeting entity of each gene drive should target a nuclease to cut or nick at a different site in the target genome such that there is no direct interaction between the nuclease activities at each target site.

Thus, each target site is preselected to be independent of the target sites for each of the other nuclease targeting entities provided by other gene drive constructs used in the present method.

Accordingly, the distance between target sites may depend on the nuclease targeting entity and nuclease to be used. Target site requirements for suitably nuclease targeting entities and corresponding nucleases are well known in the art.

Suitably, each target site may be less than 0.5 kb, less than 1 kb, less than 2 kb, less than 5 kb, or less than 10 kb apart in the genome.

### Flanking Sequence

Each gene drive construct also comprises at least two flanking sequences, one positioned on each side of the exogenous nucleotide sequence. Thus in each gene drive construct flanking sequences are positioned on opposite sides of the exogenous nucleotide sequence, such that the exogenous nucleotide sequence is between the flanking sequences. Suitably, the flanking sequences include at least a sequence which is essentially identical to a corresponding sequence in a selected target locus of the target organism.

Such flanking sequences allow a cell to insert the exogenous nucleotide sequence into its genome DNA at a cut site using well-understood mechanisms such as homologous recombination.

The flanking sequence may be any length which is suitable to allow a cell to insert the exogenous nucleotide sequence into its genome DNA at a cut site through homologous recombination or another homology-directed repair process.

Suitably, the flanking sequence may be 50 to 2000 nucleotides, 100 to 1500 nucleotides, 100 to 1000 nucleotides, 100 to 500 nucleotides or 100 to 250 nucleotides in length.

Suitably, the flanking sequences of each gene drive construct may comprise essentially identical sequences to the other gene drive constructs. In other words, the first and second flanking sequences of a first gene drive construct may comprise essentially identical sequences to the third and fourth flanking sequences of a second gene drive construct; respectively. Suitably, the flanking sequences of each gene drive construct may comprise different sequences. In other words, the first and second flanking sequences of a first gene drive construct may comprise different sequences to the third and fourth flanking sequences of a second gene drive construct; respectively.

By way of example, in the overwriting strategy of the present invention, the flanking sequences of each gene drive construct may comprise essentially identical sequences such that each pair of flanking sequences is essentially identical to the same corresponding sequence in a selected target locus. As such, successful homing by any gene drive construct will remove some or all endogenous target sites in the target locus and introduce target sites for each of the nuclease targeting entities present in the other gene drive constructs into to the target locus.

### Target Locus

“Target locus” is used herein to refer to a genomic region into which the gene drive construct is to be inserted.

The target locus comprises a target site for the nuclease targeting entity, which target site is located between nucleic acid sequences which are essentially identical to the flanking sequences of the gene drive construct (such that the cell can insert the exogenous nucleotide sequence into its genome DNA at a cut site through homologous recombination or another homology-directed repair process).

The target locus may be at least 0.5 kb, at least 1 kb, at least 2 kb, at least 5 kb or at least 10 kb in length.

The target locus may be a gene or a non-coding sequence or putatively non-functional locus.

The gene may be a protein-coding gene or a gene encoding a functional RNA (e.g. lncRNA or miRNA). The target locus may be in an exon or an intron.

The methods of the present invention are particularly advantageous for targeting a putatively non-functional locus, or locus which is not fertility- or viability-essential. These loci would not be expected to confer a fitness advantage on the individual. As such, drive-resistant deletion mutants will likely retain full fitness and would be expected to propagate throughout a population leading to loss of the gene drive.

Suitably, the target locus may be a putatively non-functional locus; a locus which is not essential for fertility or a locus which is not essential for viability.

As used herein, fitness-essential or viability-essential means that reduced function or loss of function of a gene results in a substantially lethal or sterile phenotype.

Suitably, the target locus may be a fitness-neutral locus.

Suitably, the target locus is not located on the X or Y chromosome.

### Vector

The present invention also provides a vector, or kit of vectors which comprises a gene drive construct of the invention. Such a vector may be used to introduce the nucleic acid sequence(s) or construct(s) into a host cell.

The vector may, for example, be a plasmid or a viral vector, such as a retroviral vector or a lentiviral vector, or a transposon based vector or synthetic mRNA.

The vector may be capable of transfecting or transducing a cell.

### Kit

The present invention also provides a plurality of gene drive constructs of the present invention or a kit comprising a plurality of gene drive constructs of the present invention.

### Use

The present invention further provides the use of a first and a second nucleic acid gene drive construct of the invention to introduce a first and a second gene drive into a population.

The nucleic acid gene drive construct is a gene drive construct as defined herein. The gene drive construct comprises an exogenous nucleotide sequence as defined herein.

The invention further provides the use of a third nucleic acid gene drive construct of the invention to introduce a third gene drive into the population.

The invention further provides the use of a fourth, a fifth or a sixth nucleic acid gene drive construct of the invention to introduce a fourth, fifth or sixth gene drive into the population, wherein the fourth, a fifth or a sixth nucleic acid construct is a nucleic acid construct as defined herein.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed. Suitably, the polynucleotides of the present invention are codon optimised to enable expression in a mammalian cell or insect cell, in particular a germline cell.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence or amino acid sequence includes any substitution of, variation of, modification of, replacement of, deletion of or addition of one (or more) nucleic acid(s) from or to the sequence.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Classical Multiplexing at a Neutral Site

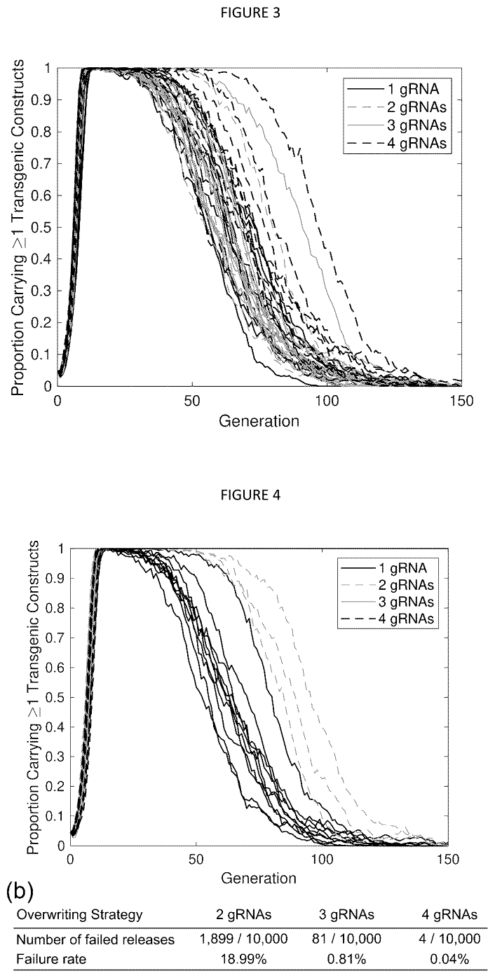

A classic multiplexing strategy was modelled whereby a single construct carries multiple gRNAs that target several, tightly linked sites on the homologous chromosome. Successful homing events result in the construct replacing the region spanning the complete set of target sites. FIG. 3 shows that the addition of extra gRNAs into a multiplexed CRISPR gene drive does not substantially improve its efficacy when targeting a neutral locus.

### Example 2—Overwriting Strategy at a Neutral Site

In the present ‘overwriting’ approach each construct will be targeted to the site encoded by its own gRNA. Successful homing reactions will restore the target sites for the other segregating multiplex constructs.

This may be achieved in a number of ways, for example through designing homology arms such that homing removes all endogenous target sites but designing each multiplex construct such that it contains the target sites for each of the other constructs. Alternatively, the locus of each construct could be offset from one another such that the homology arms of each construct represent the homologous wild-type sequence of each other construct. In both cases, successful homing by any construct will delete any existing constructs and/or resistant alleles carried on the homologous chromosome so long as all target sites are located close enough to allow constructs to target and ‘overwrite’ each other.

Since each successful homing event results in the homologous chromosome carrying only one construct, multiple simultaneous DSBs on the same chromosome (and deletion of the intervening sequence) are eliminated.

To avoid drive constructs targeting each other in the release generation (potentially altering the introduction frequencies of some constructs) n distinct pools of individuals are introduced each heterozygous for only one of the n constructs (i.e. individuals are heterozygous at only one target site, and thus it is not possible for an individual to carry two constructs on the chromosome).

Results in FIG. 4(a) demonstrate a substantial improvement over Example 1.

The majority of simulated introductions show an increase in frequency such that every individual carries at least one transgenic construct until at least 150 generations after the time of release.

Some simulated introductions fail to persist. To more accurately assess failure rates 10,000 numerical simulations were conducted for two, three and four target site cases with a population size of 1,000 (results in FIG. 4(b)). This shows a reduction in failure rates of one or more order of magnitude for each additional site targeted.

### Example 3—Blocking Strategy at a Neutral Site

Here a successful homing event by one construct removes the target sites of other segregating constructs.

The possibility of multiple simultaneous DSBs on the same chromosome is eliminated as the presence of one construct ‘blocks’ the homing action of others, preventing them becoming linked.

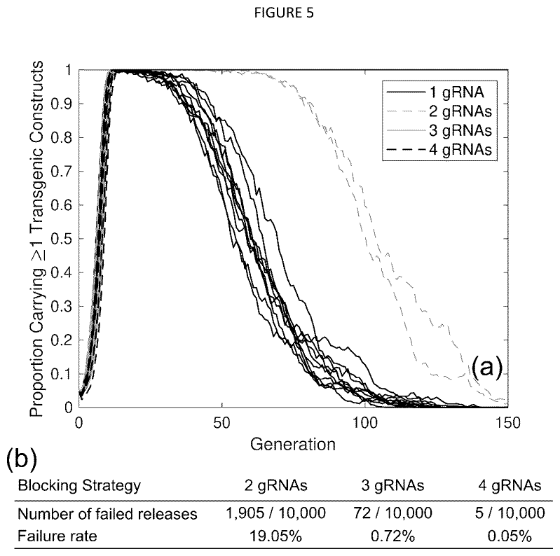

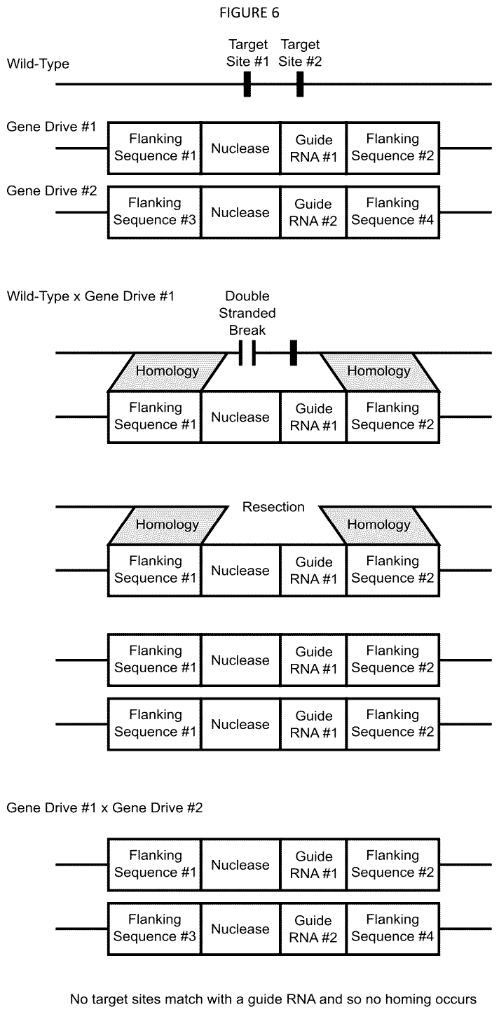

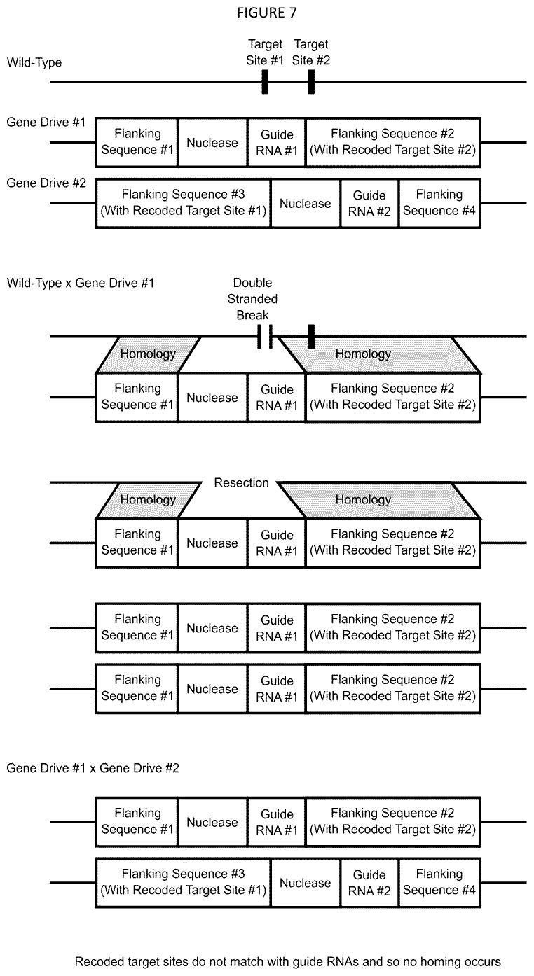

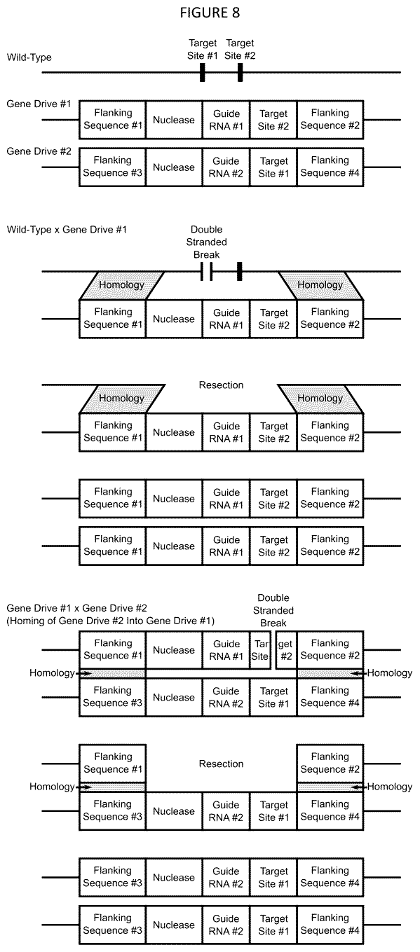

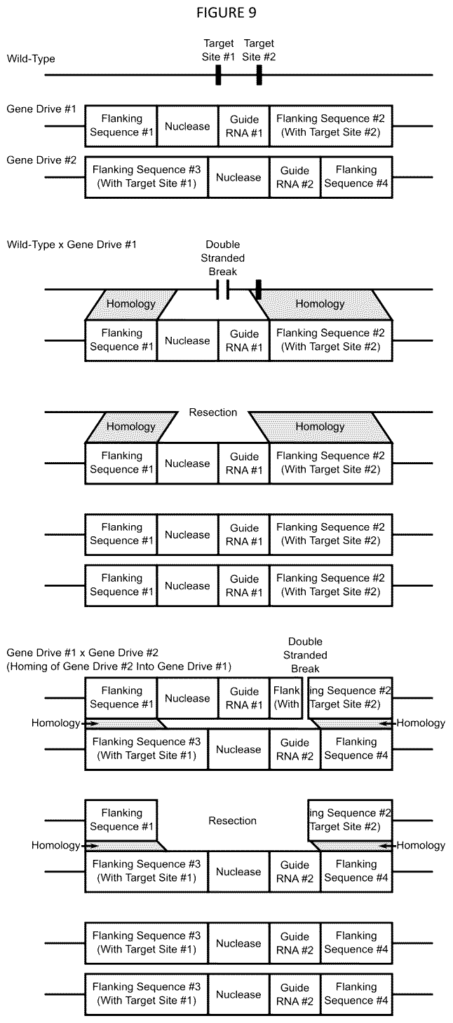

Results in FIG. 5(a) display a substantial improvement over Example 1. An additional 10,000 simulations (FIG. 5(b)) were used to assess the failure rate of this strategy, showing comparable improvements to the overwriting strategy in that adding further gRNAs gives one or more orders of magnitude reduction in the failure rate.

### Example 4—Efficacy in Larger Population

Increasing the population size presents a greater challenge for CRISPR drives as it increases opportunities for fully resistant individuals to emerge, likely raising the failure rate.

As such, Table 1 shows failure rates estimates obtained for increased population sizes using 500 numerical simulations each of populations of 10,000, 20,000, 30,000, 40,000 and 50,000 individuals, whilst all other parameters remain equal.

TABLE 1
A summary of numerical simulations conducted and the
resulting failure rates (in parentheses) for the blocking
strategy in a range of different population sizes.
Each simulation considered a release ratio of 0.05.

| | Blocking Strategy | | |
|---|---|---|---|
| Population Size | 2 gRNAs | 3 gRNAs | 4 gRNAs |
| 10,000 | 432/500 | 16/500 | 1/500 |
| | (86.40%) | (3.20%) | (0.20%) |
| 20,000 | 489/500 | 52/500 | 1/500 |
| | (97.80%) | (10.40%) | (0.20%) |
| 30,000 | 495/500 | 72/500 | 2/500 |
| | (99.00%) | (14.40%) | (0.40%) |
| 40,000 | 499/500 | 87/500 | 2/500 |
| | (99.80%) | (17.40%) | (0.40%) |
| 50,000 | 500/500 | 105/500 | 3/500 |
| | (100.00%) | (21.00%) | (0.60%) |

Failure rates rise linearly as population sizes increase with rates asymptoting as they approach 100%.

Assuming this to be the case allows the extrapolation of results in Table 1 to predict failure rates for populations that would otherwise require a large computational investment.

Considering conditions giving a 90% chance of a successful transgenic introduction (i.e. a failure rate of 10%), CRISPR drives employing 3 and 4 target blocking designs could be successfully implemented in populations of up to 20,000 and 1,000,000 individuals, respectively.

Assuming similar improvements continue to be seen from the addition of further gRNAs, it is expected that this strategy would be capable of transforming populations over extremely large geographic scales.

### Materials and Methods

### Simulated Experimental Setup

A laboratory cage experimental setup was modelled. A fixed population of 1,000 individuals with 1:1 male to female ratio was considered (unless otherwise stated). Initially, transgenic individuals were added to a wild-type population at a pre-determined ratio. Individuals were then paired for mating and offspring produced with genotypes dependent on those of the parents. For each individual, effects of each gene drive and their associated fitness costs were simulated. From resulting offspring 1,000 individuals (500 male, 500 female) were selected to seed the next generation. This was repeated for 150 generations with the transgenic frequency recorded in each.

### Parameter Values

Parameter values used for all numerical simulations are as follows. Release ratio=0.05 (for one construct or divided by n for n-target sites), probability of endonuclease cutting=0.85, NHEJ repair probability=0.02, homozygote fitness cost=0.15 and dominance of fitness cost=0.5 with fitness costs applied multiplicatively across constructs.

### Release Methodologies Simulated

Two different strategies were considered for the introduction of transgenic individuals. For the classic multiplexing individuals heterozygous at all target sites were introduced into a wild-type population. For overwriting and blocking strategies the release ratio was divided between the n target sites (rounded down to the nearest whole individual) and n separate pools of individuals heterozygous at a single target site introduced. For simplicity, the release of both males and females was assumed to be a 1:1 ratio.

### Calculation of Failure Rates

A simulated introduction of transgenic individuals was deemed to have failed if the frequency of individuals carrying one or more transgenic construct was less than 0.9 after 150 generations.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of generating a plurality of engineered subpopulations each comprising a gene drive, said method comprising:
introducing a first gene drive construct into the germline genomic DNA of a first subpopulation to produce a first engineered subpopulation, which first gene drive construct comprises a first exogenous nucleotide sequence comprising a first entity which is, or encodes, a component of a homing nuclease; which homing nuclease is capable of a targeting a first target sequence via a first targeting entity, and a first flanking sequence and a second flanking sequence, wherein at least the first exogenous nucleotide sequence is located between the first flanking sequence and the second flanking sequence;
introducing a second gene drive construct into the germline genomic DNA of a second subpopulation to produce a second engineered subpopulation, which second gene drive construct comprises a second exogenous nucleotide sequence comprising a second entity which is, or encodes, a component of a homing nuclease; which homing nuclease is capable of a targeting a second target sequence via a second targeting entity, and a third flanking sequence and a fourth flanking sequence, wherein at least the second exogenous nucleotide sequence is located between the third flanking sequence and the fourth flanking sequence;
wherein the first and second flanking sequences and the third and fourth flanking sequences are substantially identical to target regions located in the same target locus in the genomic DNA;
and wherein each of the first target sequence and the second target sequence in the genomic DNA is located between the target regions which are substantially identical to the first and second flanking sequences and the third and fourth flanking sequences or within a target region which is substantially identical to one of the first, second, third or fourth flanking sequence;
wherein
(i)
(a) the first exogenous nucleotide sequence between the first flanking sequence and second flanking sequence of the first gene drive comprises a target sequence for the second nuclease targeting entity e; and
(b) the second exogenous nucleotide sequence between the third flanking sequence and fourth flanking sequence of the second gene drive comprises a target sequence for the first nuclease targeting entity; or
(ii)
(a) the first exogenous nucleotide sequence between the first flanking sequence and second flanking sequence of the first gene drive does not comprise a target sequence for the second targeting entity; and
(b) the second exogenous nucleotide sequence between the third flanking sequence and fourth flanking sequence of the second gene drive does not comprise a target sequence for the first nuclease targeting entity.

2. A method according to claim 1, which further comprises introducing a third gene drive construct into the germline genomic DNA of a third subpopulation to produce a third engineered subpopulation, which third gene drive construct comprises a third exogenous nucleotide sequence comprising a third entity which is, or encodes, a component of a homing nuclease; which homing nuclease is capable of a targeting a third target sequence via a third targeting entity, and a fifth flanking sequence and a sixth flanking sequence;
wherein at least the third exogenous nucleotide sequence is located between the fifth flanking sequence and the sixth flanking sequence;
wherein the fifth and sixth flanking sequences are substantially identical to target regions located in the same target locus in the genomic DNA as the first and second flanking sequences and the third and fourth flanking sequences; and
wherein the target sequences of the first, second and third nuclease targeting entities are located between the target regions in the genomic DNA which are substantially identical to the first and second flanking sequences, the third and fourth flanking sequences, and the fifth and sixth flanking sequences or within a target region which is substantially identical to one of the first, second, third, fourth, fifth or sixth flanking sequence;
wherein
(i)
(a) the first exogenous nucleotide sequence between the first flanking sequence and second flanking sequence of the first gene drive comprises a target sequence for the second and third nuclease targeting entities;
(b) the second exogenous nucleotide sequence the third flanking sequence and fourth flanking sequence of the second gene drive comprises a target sequence for the first and third nuclease targeting entities; and
(c) the third exogenous nucleotide sequence between the fifth flanking sequence and sixth flanking sequence of the third gene drive comprises a target sequence for the first and second nuclease targeting entities; or
(ii)
(a) the first exogenous nucleotide sequence between the first flanking sequence and second flanking sequence of the first gene drive does not comprise a target sequence for the second and third nuclease targeting entities;
(b) the second exogenous nucleotide sequence between the third flanking sequence and fourth flanking sequence of the second gene drive does not comprise a target sequence for the first and third nuclease targeting entities; and
(c) the third exogenous nucleotide sequence between the fourth flanking sequence and fifth flanking sequence of the third gene drive does not comprise a target sequence for the first and second nuclease targeting entities.

3. The method according to claim 1 further comprising introducing a further gene drive into a fourth, fifth or sixth subpopulation to produce a fourth, fifth or sixth engineered subpopulation, wherein each further subpopulation comprises a further gene drive.

4. The method according to claim 1, further comprising introducing the engineered subpopulations into a general population.

5. The method according to claim 1, wherein the component of the homing nuclease within the exogenous nucleotide sequence is a targeting entity which is capable of a targeting a nuclease to a target sequence and/or a nuclease.

6. The method according to claim 5 wherein the entity which is capable of a targeting a nuclease to a target sequence is a polynucleotide which is capable of directing a nuclease to a target site, a polynucleotide encoding a polypeptide which is capable of directing a nuclease to a target site or a polynucleotide which encodes a target-specific nuclease.

7. The method according to claim 1 wherein the exogenous nucleotide sequence of the gene drives comprises 1000 to 15000 nucleotides.

8. The method according to claim 6 wherein the polynucleotide which directs a nuclease to a target site is a CRISPR RNA (crRNA) or guideRNA (gRNA).

9. The method according to claim 8 wherein the nuclease is a CRISPR-associated nuclease.

10. The method according to claim 1 wherein the exogenous nucleotide sequence of one or more of the gene drives further encodes a nuclease or wherein the exogenous nucleotide sequence of each of the gene drives further encodes a nuclease.

11. (canceled)

12. The method according to claim 1 wherein the distance between the target site of each nuclease targeting entity is less than 10 kb, less than 9 kb, less than 8 kb, less than 7 kb, less than 6 kb, less than 5 kb, less than 4 kb, less than 3 kb, less than 2 kb or less than 1 kb.

13. The method according to claim 1 wherein the target locus
i) is a non-coding sequence or putatively non-functional locus;
ii) is not a fertility- or viability-essential locus; and/or
iii) is a fitness-neutral locus.

14-15. (canceled)

16. The method according to claim 1 wherein the target locus is not located on the X or Y chromosome.

17. (canceled)

18. A kit comprising at least a first and a second nucleic acid construct, wherein:
(i) the first nucleic acid construct comprises a first exogenous nucleotide sequence comprising a first entity which is, or encodes, a component of a homing nuclease; which homing nuclease is capable of a targeting a first target sequence via a first targeting entity, and a first flanking sequence and a second flanking sequence; wherein at least the first exogenous nucleotide sequence is located between the first flanking sequence and the second flanking sequence; and
(ii) the second nucleic acid construct comprises a second exogenous nucleotide sequence comprising a second entity which is, or encodes, a component of a homing nuclease; which homing nuclease is capable of targeting a second target sequence via a second targeting entity, and a third flanking sequence and a fourth flanking sequence;
wherein at least the second exogenous nucleotide sequence is located between the third flanking sequence and the fourth flanking sequence wherein the first and second flanking sequences and the third and fourth flanking sequences are substantially identical to target regions located in the same target locus in the genomic DNA of a target population; and
wherein each of the first target sequence and the second target sequence in the genomic DNA is located between the target regions which are substantially identical to the first and second flanking sequences and the third and fourth flanking sequences or within a target region which is substantially identical to one of the first, second, third or fourth flanking sequence;
wherein
(i)
(a) the first exogenous nucleotide sequence between the first flanking sequence and second flanking sequence of the nucleic acid construct comprises a target sequence for the second nuclease targeting entity; and
(b) the second exogenous nucleotide sequence between the third flanking sequence and fourth flanking sequence of the second nucleic acid construct comprises a target sequence for the first nuclease targeting entity; or
(ii)
(a) the first exogenous nucleotide sequence between the first flanking sequence and second flanking sequence of the first nucleic acid construct does not comprise a target sequence for the second targeting entity; and
(b) the second exogenous nucleotide sequence between the third flanking sequence and fourth flanking sequence of the second nucleic acid construct does not comprise a target sequence for first nuclease targeting entity.

19. A kit according to claim 18 further comprising a third nucleic acid construct which comprises a third exogenous nucleotide sequence comprising a third entity which is, or encodes, a component of a homing nuclease; which homing nuclease is capable of a targeting a third target sequence via a third targeting entity, and a fifth flanking sequence and a sixth flanking sequence, wherein at least the third exogenous nucleotide sequence is located between the fifth flanking sequence and the sixth flanking sequence;
wherein the fifth and sixth flanking sequences are substantially identical to target regions located in the same target locus in the genomic DNA of the target population as the first and second flanking sequences and the third and fourth flanking sequences; and
wherein the target sequences of the first, second and third nuclease targeting entities are located between the target regions in the genomic DNA which are substantially identical to the first and second flanking sequences, the third and fourth flanking sequences, and the fifth and sixth flanking sequences or within a target region which is substantially identical to one of the first, second, third, fourth, fifth or sixth flanking sequence;
wherein
(i)
(a) the first exogenous nucleotide sequence between the first flanking sequence and second flanking sequence of the first nucleic acid construct comprises a target sequence for the second and third nuclease targeting entities;
(b) the second exogenous nucleotide sequence the third flanking sequence and fourth flanking sequence of the second nucleic acid construct comprises a target sequence for the first and third nuclease targeting entities; and
(c) the third exogenous nucleotide sequence between the fifth flanking sequence and sixth flanking sequence of the third nucleic acid construct comprises a target sequence for the first and second nuclease targeting entities t; or
(ii)
(a) the first exogenous nucleotide sequence between the first flanking sequence and second flanking sequence of the first nucleic acid construct does not comprise a target sequence for the second and third nuclease targeting entities;
(b) the second exogenous nucleotide sequence between the third flanking sequence and fourth flanking sequence of the second nucleic acid construct does not comprise a target sequence for the first and third nuclease targeting entities; and
(c) the third exogenous nucleotide sequence between the fourth flanking sequence and fifth flanking sequence of the third nucleic acid construct does not comprise a target sequence for the first and second nuclease targeting entities.

20. A kit according to claim 18 further comprising a fourth, a fifth or a sixth nucleic acid construct.

21. A kit according to claim 18 wherein the nucleic acid construct comprises an exogenous nucleotide sequence as defined in claim 5.

22. A method to introduce a first and a second gene drive into a population with a first and a second nucleic acid construct, wherein:
(i) the first nucleic acid construct comprises a first exogenous nucleotide sequence comprising a first entity which is, or encodes, a component of a homing nuclease; which homing nuclease is capable of targeting a first target sequence via a first targeting entity, and a first flanking sequence and a second flanking sequence; wherein at least the first exogenous nucleotide sequence is located between the first flanking sequence and the second flanking sequence; and
(ii) the second nucleic acid construct comprises a second exogenous nucleotide sequence comprising a second entity which is, or encodes, a component of a homing nuclease; which homing nuclease is capable of targeting a second target sequence via a second targeting entity, and a third flanking sequence and a fourth flanking sequence; wherein at least the second exogenous nucleotide sequence is located between the third flanking sequence and the fourth flanking sequence
wherein the first and second flanking sequences and the third and fourth flanking sequences are substantially identical to target regions located in the same target locus in the genomic DNA of a target population; and
wherein each of the first target sequence and the second target sequence in the genomic DNA is located between the target regions which are substantially identical to the first and second flanking sequences and the third and fourth flanking sequences or within a target region which is substantially identical to one of the first, second, third or fourth flanking sequence;
wherein
(i)
(a) the first exogenous nucleotide sequence between the first flanking sequence and second flanking sequence of the nucleic acid construct comprises a target sequence for the second nuclease targeting entity; and
(b) the second exogenous nucleotide sequence between the third flanking sequence and fourth flanking sequence of the second nucleic acid construct comprises a target sequence for the first nuclease targeting entity; or
(ii)
(a) the first exogenous nucleotide sequence between the first flanking sequence and second flanking sequence of the first nucleic acid construct does not comprise a target sequence for the second targeting entity; and
(b) the second exogenous nucleotide sequence between the third flanking sequence and fourth flanking sequence of the second nucleic acid construct does not comprise a target sequence for the first nuclease targeting entity.

23. A method according to claim 22, further comprising the use of a third nucleic acid construct to introduce a third gene drive into the population; wherein the third nucleic acid construct comprises a third exogenous nucleotide sequence comprising a third entity which is, or encodes, a component of a homing nuclease; which homing nuclease is capable of targeting a third target sequence via a third targeting entity, and a fifth flanking sequence and a sixth flanking sequence, wherein at least the third exogenous nucleotide sequence is located between the fifth flanking sequence and the sixth flanking sequence;
wherein the fifth and sixth flanking sequences are substantially identical to target regions located in the same target locus in the genomic DNA of the target population as the first and second flanking sequences and the third and fourth flanking sequences; and
wherein the target sequences of the first, second and third nuclease targeting entities are located between the target regions in the genomic DNA which are substantially identical to the first and second flanking sequences, the third and fourth flanking sequences, and the fifth and sixth flanking sequences or within a target region which is substantially identical to one of the first, second, third, fourth, fifth or sixth flanking sequence;
wherein
(i)
(a) the first exogenous nucleotide sequence between the first flanking sequence and second flanking sequence of the first nucleic acid construct comprises a target sequence for the second and third nuclease targeting entities;
(b) the second exogenous nucleotide sequence the third flanking sequence and fourth flanking sequence of the second nucleic acid construct comprises a target sequence for the first and third nuclease targeting entities; and
(c) the third exogenous nucleotide sequence between the fifth flanking sequence and sixth flanking sequence of the third nucleic acid construct comprises a target sequence for the first and second nuclease targeting entities; or
(ii)
(a) the first exogenous nucleotide sequence between the first flanking sequence and second flanking sequence of the first nucleic acid construct does not comprise a target sequence for the second and third nuclease targeting entities;
(b) the second exogenous nucleotide sequence between the third flanking sequence and fourth flanking sequence of the second nucleic acid construct does not comprise a target sequence for the first and third nuclease targeting entities; and
(c) the third exogenous nucleotide sequence between the fourth flanking sequence and fifth flanking sequence of the third nucleic acid construct does not comprise a target sequence for the first and second nuclease targeting entities.

24. A use according to claim 22 further comprising the use a fourth, a fifth or a sixth nucleic acid construct to introduce a fourth, fifth or sixth gene drive into the population.

25. (canceled)
